(19) United States
(12) Patent Application Publication
(10) Pub. No.: US 2021/0316021 A1
(43) Pub. Date: Oct. 14, 2021

(54) FOOTWEAR SANITIZING SYSTEM

(71) Applicants: Antonio Brown, N. Las Vegas, NV (US); Taraneh Jones, Henderson, NV (US)

(72) Inventors: Antonio Brown, N. Las Vegas, NV (US); Taraneh Jones, Henderson, NV (US)

(21) Appl. No.: 16/926,665

(22) Filed: Jul. 11, 2020

Related U.S. Application Data
(60) Provisional application No. 63/101,036, filed on Apr. 14, 2020.

(51) Int. Cl. A61L 2/10; A61L 2/26; A61L 2/24; A47L 23/02; A47L 23/18

(52) U.S. Cl. CPC A61L 2/10; A61L 2/26; A61L 2/24; A61L 2202/14; A47L 23/18; A61L 2202/11; A61L 2202/16; A47L 23/02

(57) ABSTRACT

A footwear sanitizing system; the footwear sanitizing system includes a sterilizing unit, a first shoe tree, a second shoe tree, and a charger working in combination to provide a sterilizing device for footwear. The footwear sanitizing system provides internal and external sanitization of footwear worn in healthcare facilities through ultraviolet radiation, preventing unwanted transfer of bacteria and germs both within and outside of the environment of use of the footwear. Users are notified of completion of the sanitization via a software application program hosted on a computing device. The footwear sanitizing system reduces the risks of nosocomial infections and healthcare-associated infections occurring within, and transferred from, healthcare facilities via a necessary fomite.

### CROSS-REFERENCE TO RELATED APPLICATION(S)

The present application is related to and claims priority to U.S. Provisional Patent Application No. 63/101,036 filed Apr. 14, 2020, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of disinfecting devices of existing art and more specifically relates to a shoe disinfecting device.

### RELATED ART

Hospitals are breeding grounds for all kinds of pathogens such as COVID-19, MRSA, bacteria, and pathogens. These pathogens often infect people at the hospital, such as visitors, workers and even patients, and make them sick. It is also common for people at hospitals to track those pathogens with them to their cars, homes, and other places. Those pathogens then contaminant those locations and put people, such as their very own family, at risk for illness. Shoes are a major culprit in spreading these germs, and the current cleaning methods, such as wiping shoes down with sanitary clothes, are not fully effective. Any person who enters a hospital, nursing home, surgery center, etc. encounters this risk. A suitable solution is desired to prevent and effectively eliminate this risk.

U.S. Pat. No. 7,960,706 to Adam Ullman relates to a shoe sanitizer. The described shoe sanitizer includes an apparatus for sanitizing human footwear having an opening in which a person's foot is inserted to put on the footwear. Introducing ultraviolet (UV) light to alter the environment inside a shoe destroys microorganisms or inhibits their growth. Visible light can also be used to prevent further growth. A preferred embodiment comprises an adjustable shoe tree equipped with a UV germicidal light source and electronic safeguards that prevent appreciable leakage of UV radiation outside the shoe.

### SUMMARY OF THE INVENTION

In view of the foregoing disadvantages inherent in the known shoe disinfecting devices art, the present disclosure provides a novel footwear sanitizing system. The general purpose of the present disclosure, which will be described subsequently in greater detail, is to provide a footwear sanitizing system for internal and external sanitization of footwear worn in healthcare facilities through ultraviolet (UV) radiation, preventing unwanted transfer of bacteria and germs both within and outside of the environment of use of the footwear.

A footwear sanitizing system is disclosed herein. The footwear sanitizing system includes a sanitizing assembly comprising a box-like sterilizing unit configured to sanitize an exterior portion of footwear inserted within. The sterilizing unit in a preferred embodiment includes a hinged lid hosting a first-removable tray supporting a first shortwave-range ultraviolet light member, a base hosting a second-removable tray supporting a second shortwave-range ultraviolet light member, and a preferably rechargeable battery source. The lid also includes an electronic locking mechanism. The first-removable tray and the second-removable tray each comprise a reflective coating configured to amplify an intensity of light emitted from the first UV-C light member and the second UV-C light member respectively.

The sanitizing assembly further comprises a first shoe tree for insertion in a first-shoe and a second shoe tree for insertion in a second-shoe. The first shoe tree and the second shoe tree each feature a shoe tree shortwave-range ultraviolet light configured to emit UV-C light in order to sanitize an interior portion of the first-shoe and the second-shoe. In combination, the sterilizing unit, the first shoe tree, and the second shoe tree provide means for sterilizing the interior and exterior portions of footwear.

The footwear sanitizing system further comprises a software application program downloadable to an existing computing device in wireless communication with the sanitizing assembly. The software application program allows a user to remotely control and monitor the sanitizing assembly. The box-like sterilizing unit, the first shoe tree and the second shoe tree are in wireless communication with each other and the sanitizing assembly provides a footwear sanitizing system for sanitizing footwear inserted within and eliminating germs, pathogens and contaminants collected by the footwear by utilizing a plurality of UV-C lights.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a footwear sanitizing system, constructed and operative according to the teachings of the present disclosure.

FIG. 1 is a perspective view of the footwear sanitizing system during an ‘in-use’ condition, according to an embodiment of the disclosure.

FIG. 2 is a perspective view of the footwear sanitizing system of FIG. 1 having a sanitizing assembly including a box-like sterilizing unit, a first shoe tree, a second shoe tree, and a charger, according to an embodiment of the present disclosure.

FIG. 3 is a perspective view of the footwear sanitizing system of FIG. 1 comprising the first shoe tree and the second shoe tree, according to an embodiment of the present disclosure.

FIG. 4 is a perspective view of the second shoe tree of the footwear sanitizing system of FIG. 1, according to an embodiment of the present disclosure.

FIG. 5 is a top perspective view of the sterilizing unit of the footwear sanitizing system of FIG. 1, according to an embodiment of the present disclosure.

FIG. 6 is a side perspective view of the sterilizing unit of the footwear sanitizing system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a shoe disinfecting device and more particularly to a footwear sanitizing system as used to improve the means for sanitizing items using UV-C lights. The footwear sanitizing system is particularly useful for healthcare professionals. Persons who work in healthcare environments commonly walk through and on germs, bacteria, viruses and other pathogens, thus subjecting their footwear to accumulation of such germs, the footwear sanitizing system disinfects footwear from the germs. The footwear sanitizing system effectively eliminates a risk of trans-environment infection. The footwear sanitizing system can benefit environments of other occupational fields, as well, such as but not limited to medical and pharmaceutical laboratories, processed food production plants, beverage bottling facilities, butcheries and stockyards, animal hatcheries and nurseries, dairy farms, sanitation facilities, water treatment plants, and other occupational environments of relativity.

Generally, the footwear sanitizing system utilizes broadcast shortrange ultraviolet (UV-C) light to disinfect footwear on both an exterior and an interior of footwear. The shortrange ultraviolet (UV-C) light of the footwear sanitizing system deactivates the deoxyribonucleic acid (DNA) of germs, bacteria, viruses, and other pathogens. Additionally, the shortrange ultraviolet (UV-C) light of the footwear sanitizing system prevents germs, bacteria, viruses, and other pathogens from multiplying and replicating. By preventing their multiplication and replication, the footwear sanitizing system causes germs, bacteria, viruses and other pathogens to die. By exterminating germs, bacteria, viruses and other pathogens from footwear, the footwear sanitizing system eliminates the risk of the germs from infecting other environments in which the footwear may be present.

More specifically, the footwear sanitizing system features a sterilizing unit into which footwear may be inserted, ultraviolet lights of shortwave range (UV-C) capacity that broadcasts upon the exterior of the footwear, and two UV-C shoe tree units that individually apply within the interior of the footwear. Each shoe tree features UV-C lights and an internal rechargeable battery for its powering.

Additionally, an internal and electrical processing component is featured for wired connection between the shortwave-range ultraviolet lights (UV-Cs) and a sealing latch of the sterilizing unit, requiring the latch to be closed to continue electrical current needed to activate and operate the UV-Cs. The processing component also features a timing program for discontinuance of operation after completion of a sanitizing cycle. Proximity sensors are featured in each of the two UV-C shoe tree units and communicate with the processing component of the sterilizing unit. An inductive power transfer (IPT) device for wireless recharging of the batteries of the footwear sanitizing system is featured Preferably, the charger is provided in a format for application into and broadcast from a power socket of a motor vehicle.

A power button and a display screen are featured on the exterior of the sterilizing unit to display progress of the disinfection cycle. In a preferred embodiment, the sterilizing unit features a fabric pocket an exterior sidewall of the sterilizing unit for storage of other footwear a user may wear during use of device.

A software application program, for download upon and use by another device, such as but not limited to a tablet or smartphone, is also provided with the footwear sanitizing system to broadcast alert of sanitizing cycle completion as well as other notifications.

In a most preferred embodiment, the sterilizing unit of the footwear sanitizing system is made of carbon fiber in a rectangular shape, measuring approximately sixteen inches in length by twelve inches in width by eight inches in height (16″×12″×8″). A covering lid attaches to a sidewall by hinges. The footwear sanitizing system can be made in various sizes and shapes and various applicable materials. A projecting tab extends from the lower plane of a sidewall of this lid and into a corresponding slot within the base unit. This contact of the tab within the slot completes the circuit needed for operation of an internal electrical processing component. This electrical processing component connects by wire to shortwave-range ultraviolet lights (UV-Cs), which are featured within the floor of the sterilizing unit and within the interior of the lid. A reflective coating is included behind the lights to increase intensity. The UV-Cs are contained within retractable trays that are covered by durable transparent plexiglass. These UV-Cs broadcast at approximately two hundred and sixty nanometers (260 nλ). An internal and rechargeable battery is included in the base of the sterilizing unit for powering of these UV-Cs, as well as other electronic components of the sterilizing unit.

Two (2) additional shortwave-range ultraviolet light (UV-C) are in the form of shoe tree inserts and the UV-C lighting broadcasts through the toe, arch and heel areas. These additional UV-Cs also broadcast at approximately two hundred and sixty nanometers (260 nλ). Each of these UV-C units for shoe insertion are powered by internal and rechargeable batteries and include proximity sensors that communicate with the electrical processing component, allowing their activation only when within the sterilizing unit. On the exterior of the lid is a light-emitting diode (LED) screen, which displays the time of operation as well as battery-life status. Next to this display screen is a power button. An inductive power transfer (IPT) device, using resonant inductive coupling of mid-range broadcast range, is featured within a separate structure for the remote recharging of batteries within the sterilizing unit and the shoe tree inserts. This IPT includes a plug for its insertion within a motor vehicle power socket, and includes a display screen that indicates its activity.

A preferred method of use is as follows: A user may remove his or her footwear upon exiting a healthcare environment or other location, and may apply another pair of shoes that can be stored within the storage pouch. The user may then insert a first shoe tree into a removed first-shoe, insert a second shoe tree into a removed second-shoe, and then place the footwear within the sanitizing unit. The lid of the sanitizing unit may then be closed and sealed, and the device may be turned on. The display screen of the footwear sanitizing system will report the status of the shoe sanitization, as well as battery-life status. The user may also consult his or her personal smartphone or tablet to view the progress via the software application program of the footwear sanitizing system, “Box N Out.” Should the batteries of the footwear sanitizing system need recharging, the user may insert the inductive power transfer (IPT) device within the power socket of the motor vehicle to activate such recharging. The IPT will indicate status of the recharging.

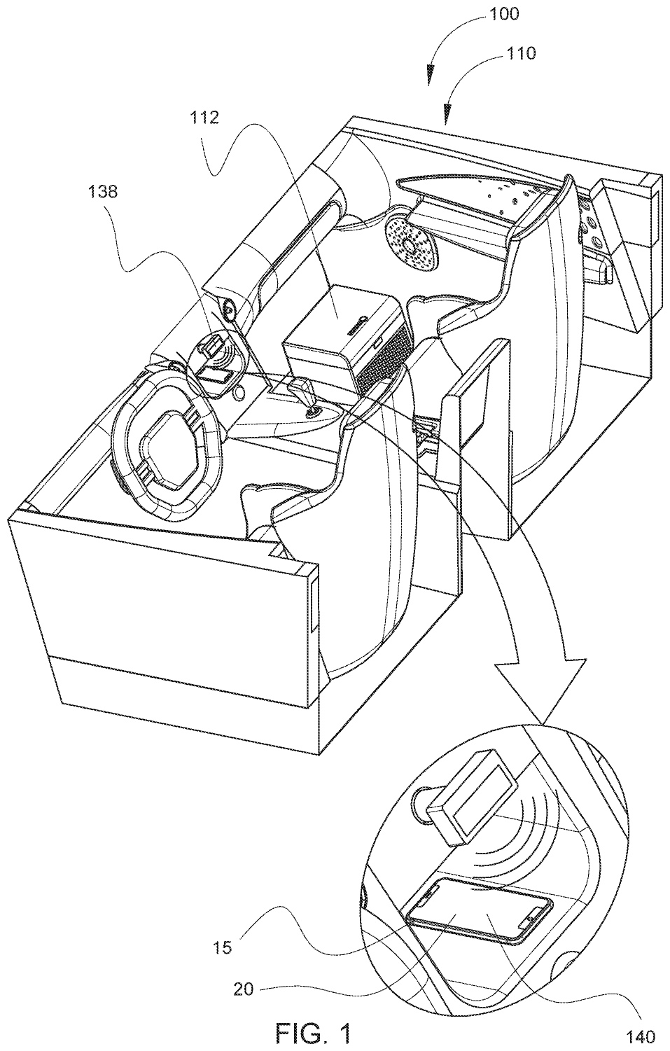

Referring now more specifically to the drawings by numerals of reference, there is shown in FIGS. 1-6, various views of a footwear sanitizing system 100.

FIG. 1 shows a footwear sanitizing system 100 during an ‘in-use’ condition, according to an embodiment of the present disclosure. Here, the footwear sanitizing system 100 may be beneficial for use by a user to eliminate germs, bacteria and pathogens on footwear. As illustrated, the footwear sanitizing system 100 may include a sanitizing assembly 110 comprising a box-like sterilizing unit 112 configured to sanitize an exterior portion of footwear inserted within. The sterilizing unit 112 comprises a hinged lid 114 hosting a first-removable tray 116 supporting a first shortwave-range ultraviolet light member 118, a base 122 hosting a second-removable tray 124 supporting a second shortwave-range ultraviolet light member 126, and a battery source 128. The lid 114 further includes an electronic locking mechanism 120. The first-removable tray 116 and the second-removable tray 124 each comprise a reflective coating 160, such as aluminum or other relevant reflectant, configured to amplify an intensity of light emitted from the first UV-C light member and the second UV-C light member respectively.

The sanitizing assembly 110 further includes a first shoe tree 130 for insertion in a first-shoe 5 and a second shoe tree 132 for insertion in a second-shoe 10. The first shoe tree 130 and the second shoe tree 132 each feature a shoe tree shortwave-range ultraviolet light 134 configured to emit UV-C light in order to sanitize an interior portion of the first-shoe 5 and the second-shoe 10. Additionally, the first shoe tree 130 and the second shoe tree 132 each comprise an internal battery 136 configured to power the first shoe tree 130 and the second shoe tree 132 respectively. A charger 138 is also provided. The charger 138 is configured to be inserted within an auxiliary power receptacle and charge the battery source 128 of the box-like sterilizing unit 112 and the internal battery 136 of the first shoe tree 130 and the second shoe tree 132. The first shortwave-range ultraviolet light member 118, the second shortwave-range ultraviolet light member, and the shoe tree shortwave-range ultraviolet lights 134 each emit at approximately two hundred and sixty nanometers (260 nλ). The ultraviolet (UV) lights of the footwear sanitizing system can broadcast at any point of the shortwave range of one hundred to two hundred and eighty nanometers (UV-C100-280 nλ) that is deemed capable of disinfecting all relevant bacteria.

The footwear sanitizing system also includes a software application program 140 downloadable to an existing computing device 15 in wireless communication with the sanitizing assembly 110 and includes a graphical user interface 20. The computing device 15 is configured to receive data regarding the sanitizing assembly 110 and display the data on the graphical user interface 20 such as sanitization cycle status, a battery life of the internal battery 136 and the battery source 128.

The box-like sterilizing unit 112, the first shoe tree 130 and the second shoe tree 132 are in wireless communication with each other and the sanitizing assembly 110 provides a footwear sanitizing system for sanitizing footwear inserted within and eliminating germs, pathogens and contaminants collected by the footwear by utilizing a plurality of UV-C lights. The footwear sanitizing system also eliminates common odors that result from presence of bacteria and other unwanted agents inside of shoes.

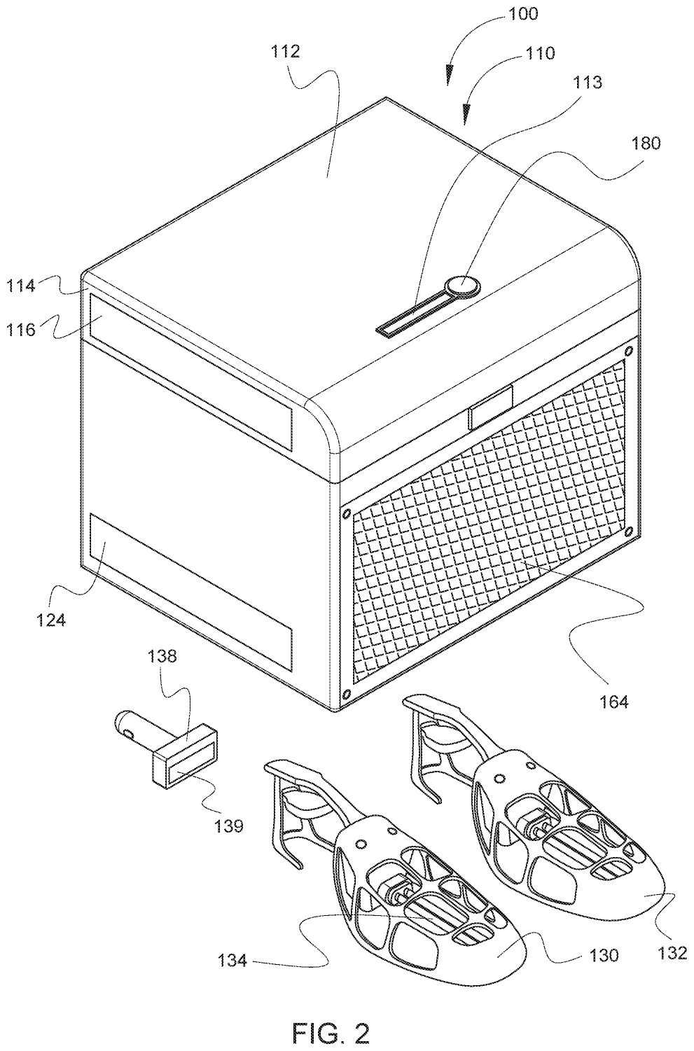

FIG. 2 shows the footwear sanitizing system 100 of FIG. 1, according to an embodiment of the present disclosure. The footwear sanitizing system 100 may be arranged as a kit. In particular, the footwear sanitizing system 100 may further include a set of instructions. The instructions may detail functional relationships in relation to the structure of the footwear sanitizing system 100 such that the footwear sanitizing system 100 can be used, maintained, or the like, in a preferred manner.

The footwear sanitizing system 100 may be powered by batteries that may or may not be of rechargeable variety, by direct current (DC) connector, and/or by alternating current (AC) power cord. The footwear sanitizing system 100 may also be capable of use with both DC connector and AC power cord, and with separate, attachable cords of specific relativity that attach to a port on the sterilizing unit 112. The battery recharging mechanism of the footwear sanitizing system 100 may or may not be of inductive power transfer (IPT) format. The charger 138 of the sanitizing assembly 110, as shown in the figures, is preferably wireless and is an inductive power transfer device transmitter which utilizes resonant inductive coupling of mid-range broadcast range providing a wireless charging source for recharging the battery source 128 of the box-like sterilizing unit 112 and the internal battery 136 of the first shoe tree 130 and the second shoe tree 132. The inductive power transfer device transmitter further includes a screen 139. The screen 139 is configured to display and indicate activity of the sanitizing assembly 110 during use.

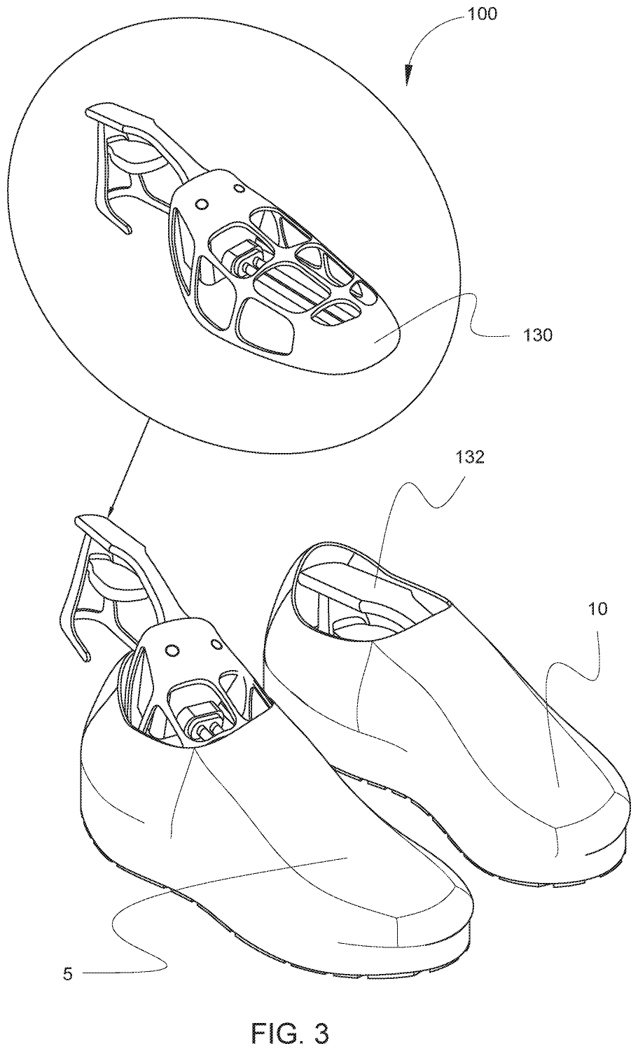

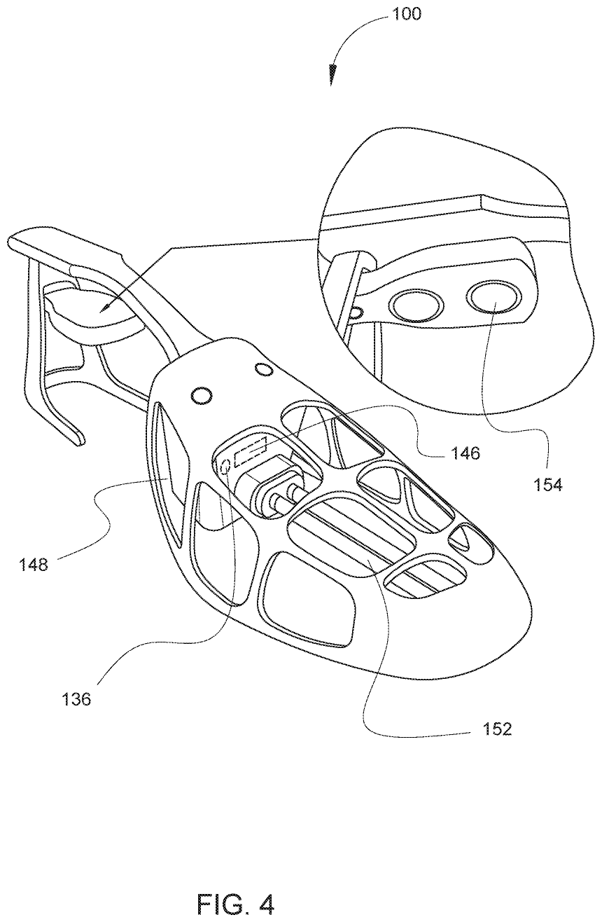

FIGS. 3-4 are perspective views of the footwear sanitizing system 100 of FIG. 1, according to an embodiment of the present disclosure. The footwear sanitizing system includes the first shoe tree 130 and the second shoe tree 132 in wireless communication with the sterilizing unit 112 and the charger 138. The first shoe tree 130 and the second shoe tree 132 each include a shoe tree shortwave-range ultraviolet light 134 configured to emit UV-C light as mentioned above. The first shortwave-range ultraviolet light member 118 and the shortwave-range ultraviolet light member are preferably removable and replaceable. The shoe tree shortwave-range ultraviolet light 134 of the first shoe tree 130 and the second shoe tree 132 includes a toe-light 152 and a heel-light 154. The toe-light 152 is positioned at a toe section and the heel-light 154 being positioned at a heel section of the first shoe tree 130 and the second shoe tree 132, as illustrated. In other alternative embodiments, the first shoe tree 130 and the second shoe tree 132 are directly connected by wire to the sterilizing unit 112. The first shoe tree 130 and the second shoe tree 132 that are inserted within footwear also function as traditional shoe trees, and thus protect the comfort, fit and appearance of the footwear.

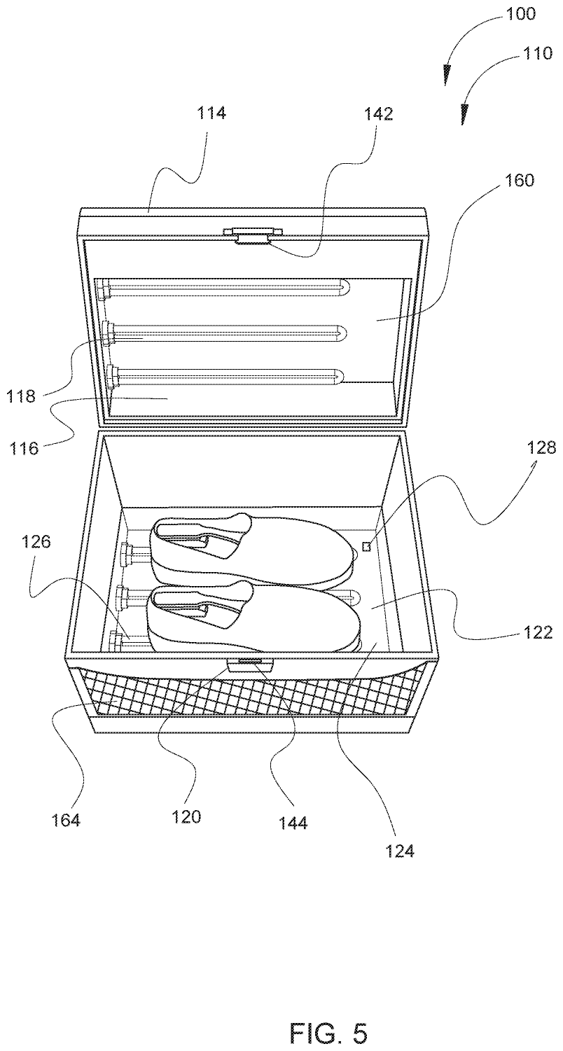

As shown in FIG. 5, the footwear sanitizing system 100 is illustrated including the sterilizing unit 112 featuring the hingedly connected lid 114 further including the electronic locking mechanism 120. The electronic locking mechanism 120 comprises a projecting tab 142 extending downwardly from a front portion of the lid 114. The projecting tab 142 is in alignment with a corresponding slot 144 at a top edge of the 122 of the sterilizing unit 112. Activation of the first shortwave-range ultraviolet light member 118 and the second shortwave-range ultraviolet light member 126 is only enabled when the projecting tab 142 is fully inserted and engaged with the corresponding slot 144 in order to prevent UV-exposure during a non-latched condition. Additionally, the first shoe tree 130 and the second shoe tree 132 each further comprise at least one proximity sensor 146 in communication with the sterilizing unit 112. Activation of the shoe tree shortwave-range ultraviolet light 134 of the first shoe tree 130 and the second shoe tree 132 is enabled only when the at least one proximity sensor 146 detects that the first shoe tree 130 and the second shoe tree 132 are within the sterilizing unit 112. The at least one proximity sensor 146 and the internal battery 136 of the first shoe tree 130 and the second shoe tree 132 are housed within a housing-unit 148, as further shown in FIG. 4. By ensuring operation only when its lid 114 is closed and sealed, the footwear sanitizing system protects its user from exposure of the ultraviolet (UV) lighting.

The sterilizing unit 112 is sized to receive and contain within an inner volume at least one set of footwear including the first-shoe 5 and the second-shoe 10 for sanitization of footwear placed within. The footwear sanitizing system 100 can be made in variations that can accommodate multiple pairs of footwear simultaneously. Additionally, the footwear sanitizing system 100 can be made in variations that treat and disinfect other medical facility items that bear fomite classification, such as but not limited to blood pressure cuffs and stethoscopes.

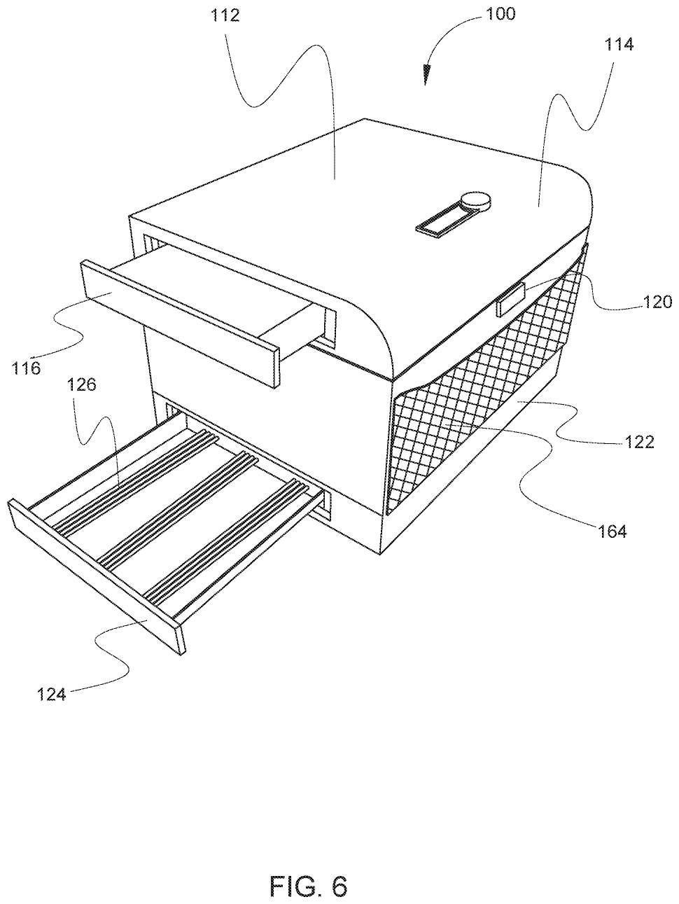

FIG. 6 is a side perspective view of the footwear sanitizing system 100, according to an embodiment of the present disclosure. The hinged lid 114 housing the first-removable tray 116 supporting the first shortwave-range ultraviolet light member 118 and the base 122 hosting the second-removable tray 124 supporting the second shortwave-range ultraviolet light member 126 are shown. The first-removable tray 116 and the second-removable tray 124 may be slidably engaged and removed from the sterilizing unit 112. The sterilizing unit 112 further includes a flexible, elasticized mesh material storage pouch 164 positioned on an exterior sidewall of the sterilizing unit 112. The storage pouch 164 is provided for safe storage of extra footwear. The sterilizing unit 112 further comprises a display screen 113 and user control interface including a power button 180. The display screen 113 of the footwear sanitizing system may be of light-emitting diode (LED) or liquid crystal display (LCD) format. The footwear sanitizing system can be made in variations that include no display screen 113, and in which the device would simply include a power light that displays a particular color when its cycle is complete. Additional user controls and buttons may be provided.

In certain embodiments, the sanitizing assembly 110 further includes a timer system configured to activate and alternatively deactivate the sanitizing assembly 110 based on set parameters. The timer system may be controllable using the software application program 140 downloadable to the existing computing device 15. The timing program of the footwear sanitizing system 100 limits its operation to only a needed period. The timing program of the footwear sanitizing system 100 reduces wear on the rechargeable batteries of the device. Additionally, the timing program of the footwear sanitizing system 100 prevents overexposure of ultraviolet lighting upon the footwear being sanitized. The footwear sanitizing system 100 may also include an audio device on its sterilizing unit 112, which will sound when the disinfection cycle is complete.

The footwear sanitizing system 100 can provide user notification of disinfection completion via various methods of wireless communication protocol, such as but not limited to Bluetooth™ and wireless local area networking. The wireless communication of the footwear sanitizing system 100 may simply be a software application program 140 that operates as a timer, set to countdown throughout a predetermined time period deemed necessary to complete footwear disinfection, and which will alert the user through his or her smartphone or tablet when the time period is complete. The footwear sanitizing system 100 may also be made in variations that include no such protocol, app, or any other wireless notification.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A footwear sanitizing system, the footwear sanitizing system comprising:
a sanitizing assembly including;
a box-like sterilizing unit configured to sanitize an exterior portion of footwear inserted within including;
a hinged lid hosting a first-removable tray supporting a first shortwave-range ultraviolet light member, said lid further including an electronic locking mechanism;
a base hosting a second-removable tray supporting a second shortwave-range ultraviolet light member;
a battery source;
a first shoe tree for insertion in a first-shoe and a second shoe tree for insertion in a second-shoe, said first shoe tree and said second shoe tree each comprising a shoe tree shortwave-range ultraviolet light configured to emit UV-C light in order to sanitize an interior portion of said first-shoe and said second-shoe, and said first shoe tree and said second shoe tree each comprise an internal battery configured to power a respective said first shoe tree and said second shoe tree; and
a charger, said charger is configured to be inserted within an auxiliary power receptacle and charge said battery source of said box-like sterilizing unit and said internal battery of said first shoe tree and said second shoe tree; and
a software application program downloadable to an existing computing device in wireless communication with said sanitizing assembly and having a graphical user interface, said computing device being configured to receive data regarding said sanitizing assembly and display said data on said graphical user interface;
wherein said first-removable tray and said second-removable tray each comprise a reflective coating configured to amplify an intensity of light emitted from said first UV-C light member and said second UV-C light member respectively; and
wherein said box-like sterilizing unit, said first shoe tree and said second shoe tree are in wireless communication with each other and said sanitizing assembly provides a footwear sanitizing system for sanitizing footwear inserted within and eliminating germs, pathogens and contaminants collected by said footwear by utilizing a plurality of UV-C lights.

2. The footwear sanitizing system of claim 1, wherein said charger is wireless and includes an inductive power transfer device transmitter which utilizes resonant inductive coupling of mid-range broadcast range providing a wireless charging source for recharging said battery source of said box-like sterilizing unit and said internal battery of said first shoe tree and said second shoe tree.

3. The footwear sanitizing system of claim 1, wherein said inductive power transfer device transmitter further includes a screen, said screen being configured to display and indicate activity of said sanitizing assembly during use.

4. The footwear sanitizing system of claim 1, wherein said electronic locking mechanism comprises a projecting tab extending downwardly from a front portion of said lid, said projecting tab is in alignment with a corresponding slot at a top edge of said base of said sterilizing unit.

5. The footwear sanitizing system of claim 4, wherein activation of said first shortwave-range ultraviolet light member and said second shortwave-range ultraviolet light member is enabled when said projecting tab is fully inserted and engaged with said corresponding slot in order to prevent UV-exposure during a non-latched condition.

6. The footwear sanitizing system of claim 5, wherein said first shoe tree and said second shoe tree each further comprise at least one proximity sensor in communication with said sterilizing unit.

7. The footwear sanitizing system of claim 6, wherein activation of said shoe tree shortwave-range ultraviolet light of said first shoe tree and said second shoe tree is enabled only when said at least one proximity sensor detects that said first shoe tree and said second shoe tree are within said sterilizing unit.

8. The footwear sanitizing system of claim 7, wherein said at least one proximity sensor and said internal battery of said first shoe tree and said second shoe tree are housed within a housing-unit.

9. The footwear sanitizing system of claim 1, wherein said first shortwave-range ultraviolet light member and said shortwave-range ultraviolet light member are removable and replaceable.

10. The footwear sanitizing system of claim 1, wherein said sterilizing unit is sized to receive and contain within an inner volume at least one set of said footwear including said first-shoe and said second-shoe.

11. The footwear sanitizing system of claim 1, wherein said first shortwave-range ultraviolet light member, said second shortwave-range ultraviolet light member, and said shoe tree shortwave-range ultraviolet lights each emit at approximately two hundred and sixty nanometers (260 nλ).

12. The footwear sanitizing system of claim 1, wherein said shoe tree shortwave-range ultraviolet light of said first shoe tree and said second shoe tree includes a toe-light and a heel-light, said toe-light being positioned at a toe section and said heel-light being positioned at a heel section.

13. The footwear sanitizing system of claim 1, wherein said sanitizing assembly further includes a timer system configured to activate and alternatively deactivate said sanitizing assembly based on set parameters.

14. The footwear sanitizing system of claim 13, wherein said timer system is controllable using said software application program downloadable to said existing computing device.

15. The footwear sanitizing system of claim 1, wherein said sterilizing unit further includes a storage pouch positioned on an exterior sidewall of the sterilizing unit.

16. The footwear sanitizing system of claim 15, wherein said storage pouch comprises a flexible, elasticized mesh material.

17. The footwear sanitizing system of claim 1, wherein said sterilizing unit further comprises a display screen and user control interface including a power button.

18. The footwear sanitizing system of claim 1, wherein said data regarding said sanitizing assembly includes a sanitization cycle status and a battery life of said internal battery and said battery source.

19. A footwear sanitizing system, the footwear sanitizing system comprising:
a sanitizing assembly including;
a box-like sterilizing unit configured to sanitize an exterior portion of footwear inserted within including;
a hinged lid hosting a first-removable tray supporting a first shortwave-range ultraviolet light member, said lid further including an electronic locking mechanism; and
a base hosting a second-removable tray supporting a second shortwave-range ultraviolet light member;
a battery source;
a first shoe tree for insertion in a first-shoe and a second shoe tree for insertion in a second-shoe, said first shoe tree and said second shoe tree each comprising a shoe tree shortwave-range ultraviolet light configured to emit UV-C light in order to sanitize an interior portion of said first-shoe and said second-shoe, and said first shoe tree and said second shoe tree each comprise an internal battery configured to power a respective said first shoe tree and said second shoe tree; and
a charger, said charger is configured to be inserted within an auxiliary power receptacle and charge said battery source of said box-like sterilizing unit and said internal battery of said first shoe tree and said second shoe tree; and
a software application program downloadable to an existing computing device in wireless communication with said sanitizing assembly and having a graphical user interface, said computing device being configured to receive data regarding said sanitizing assembly and display said data on said graphical user interface;
wherein said first-removable tray and said second-removable tray each comprise a reflective coating configured to amplify an intensity of light emitted from said first UV-C light member and said second UV-C light member respectively;
wherein said box-like sterilizing unit, said first shoe tree and said second shoe tree are in wireless communication with each other and said sanitizing assembly provides a footwear sanitizing system for sanitizing footwear inserted within and eliminating germs, pathogens and contaminants collected by said footwear by utilizing a plurality of UV-C lights;
wherein said charger is wireless and includes an inductive power transfer device transmitter which utilizes resonant inductive coupling of mid-range broadcast range providing a wireless charging source for recharging said battery source of said box-like sterilizing unit and said internal battery of said first shoe tree and said second shoe tree;
wherein said inductive power transfer device transmitter further includes a screen, said screen being configured to display and indicate activity of said sanitizing assembly during use;
wherein said electronic locking mechanism comprises a projecting tab extending downwardly from a front portion of said lid, said projecting tab is in alignment with a corresponding slot at a top edge of said base of said sterilizing unit;
wherein activation of said first shortwave-range ultraviolet light member and said second shortwave-range ultraviolet light member is enabled when said projecting tab is fully inserted and engaged with said corresponding slot in order to prevent UV-exposure during a non-latched condition;
wherein said first shoe tree and said second shoe tree each further comprise at least one proximity sensor in communication with said sterilizing unit;
wherein activation of said shoe tree shortwave-range ultraviolet light of said first shoe tree and said second shoe tree is enabled only when said at least one proximity sensor detects that said first shoe tree and said second shoe tree are within said sterilizing unit;
wherein said at least one proximity sensor and said internal battery of said first shoe tree and said second shoe tree are housed within a housing-unit;
wherein said first shortwave-range ultraviolet light member and said shortwave-range ultraviolet light member are removable and replaceable;
wherein said sterilizing unit is sized to receive and contain within an inner volume at least one set of said footwear including said first-shoe and said second-shoe;
wherein said first shortwave-range ultraviolet light member, said second shortwave-range ultraviolet light member, and said shoe tree shortwave-range ultraviolet lights each emit at approximately two hundred and sixty nanometers (260 nλ);
wherein said shoe tree shortwave-range ultraviolet light of said first shoe tree and said second shoe tree includes a toe-light and a heel-light, said toe-light being positioned at a toe section and said heel-light being positioned at a heel section;
wherein said sanitizing assembly further includes a timer system configured to activate and alternatively deactivate said sanitizing assembly based on set parameters;
wherein said timer system is controllable using said software application program downloadable to said existing computing device;
wherein the sterilizing unit further includes a storage pouch positioned on an exterior sidewall of the sterilizing unit;
wherein said storage pouch comprises a flexible, elasticized mesh material;
wherein said sterilizing unit further comprises a display screen and user control interface including a power button; and
wherein said data regarding said sanitizing assembly includes a sanitization cycle status and a battery life of said internal battery and said battery source.

20. The footwear sanitizing system of claim 19, further comprising set of instructions; and
wherein the footwear sanitizing system is arranged as a kit.
